(19) United States
(12) Patent Application Publication
(10) Pub. No.: US 2023/0130925 A1
(43) Pub. Date: Apr. 27, 2023

(54) METHODS TO PREVENT, AMELIORATE AND TREAT COMPLICATIONS FROM VIRAL INFECTIONS

(71) Applicant: University of Southern California, Los Angeles, CA (US)

(72) Inventor: Preet M. Chaudhary, Toluca Lake, CA (US)

(21) Appl. No.: 17/912,009

(22) PCT Filed: Mar. 16, 2021
(86) PCT No.: PCT/US2021/022643

§ 371 Date: Sep. 15, 2022

Related U.S. Application Data
(60) Provisional application No. 62/990,396, filed on Mar. 16, 2020.
(60) Provisional application No. 63/000,008, filed on Mar. 26, 2020.

(51) Int. Cl. A61K 31/506; A61K 31/5025; A61K 31/496; C07K 16/28; C07K 16/24; A61K 38/17; A61K 31/7076; C07K 16/18; A61K 45/06; A61K 31/513; A61K 31/675; A61K 31/4965; A61K 31/215; A61K 31/7056; A61K 31/53; A61K 31/4706; A61P 31/14; A61P 11/00; A61P 31/16; A61K 39/395

(52) U.S. Cl. CPC A61K 31/506; A61K 31/5025; A61K 31/496; C07K 16/2866; C07K 16/248; A61K 38/1793; A61K 31/7076; C07K 16/18; A61K 45/06; A61K 31/513; A61K 31/675; A61K 31/4965; A61K 31/215; A61K 31/7056; A61K 31/53; A61K 31/4706; A61P 31/14; A61P 11/00; A61P 31/16; A61K 39/3955; C07K 2317/24; C07K 2317/21; C07K 2319/30; A61K 2039/505

(57) ABSTRACT

The present disclosure relates to methods for preventing, ameliorating or treating complications caused by a viral infection (e.g., SARS, SARS-cov2, MERS, Influenza etc.), by administration of an immune response modulating agent (IRMA).

### CROSS REFERENCE TO RELATED APPLICATIONS

This application claims priority under 35 U.S.C. § 119 from U.S. Provisional Application Ser. No. 62/990,396, filed Mar. 16, 2020, and U.S. Provisional Application No. 62/000,008, filed Mar. 26, 2020, the disclosures of which are incorporated herein by reference.

### FIELD OF THE INVENTION

The present disclosure relates to methods for preventing, ameliorating or treating complications caused by a viral infection (e.g., SARS, SARS-cov2, MERS, Influenza etc.), by administration of an immune response modulating agent (IRMA). The method involves administration to a subject with a viral infection a tyrosine kinase inhibitor (e.g., a Src kinase inhibitor, e.g., Lck inhibitor, e.g., Dasatinib, Ponatinib or Bosutinib), a cytokine or a cytokine receptor antagonist (e.g., tocilizumab, sarilumab, siltuxiamab, anakinra etc.), a C5 inhibitor (e.g., tesidolumab, or eculizumab), a steroids (e.g., dexamethasone, prednisone, etc.) or a combination. In some embodiments, the viral infection is a respiratory tract infection (e.g., coronaviruses, e.g., SARS-cov2, COVID-19, SARS virus and MERS virus, RSV, influenza, parainfluenza, etc.). Features of the methods, including the timing of the administration of the agents or treatments for prevention of complications, provide various advantages, such as lower morbidity, less ICU admissions, less need for ventilatory support and improvement in survival.

### BACKGROUND

A number of viral infections (e.g. rhino-virus, adenoviruses, influenza A, influenza B, parainfluenza virus, human respiratory syncytial virus, human coronavirus (e.g., SARS, COVID-19 and MERS), enteroviruses and human metapneumovirus) are associated with complications such as Adult Respiratory Distress syndrome which require ventilatory support and can be fatal. The present disclosure provides methods and composition for the prevention and treatment of such viral infections (e.g., rhino-virus, adenoviruses, influenza A, influenza B, parainfluenza virus, human respiratory syncytial virus, human coronavirus (e.g., SARS, SARS-co2/COVID-19 and MERS), enteroviruses and human metapneumovirus). The method involves administration to a subject with a viral infection a tyrosine kinase inhibitor, a cytokine antagonist (e.g., Tocilizumab, siltuxiamab, anakinra etc.), a C5 inhibitor (e.g., tesidolumab, or eculizumab), steroids (e.g., dexamethasone, prednisone, etc.) or a combination.

### SUMMARY

Provided in some aspects are methods of prevention and treatment including administering to a subject an Immune response modulating agent (IRMA or agent) or other treatment capable of treating, preventing, delaying, or attenuating the development of a complications secondary to a viral infection. In some embodiments, the administration of the IRMA (e.g., dasatinib, ponatinib, bosutinib, tocilizumab, siltuximab etc.) or other treatment (e.g., antiviral) is at a time that is less than or no more than ten, seven, six, five, four, three, two days, 1 day after development of signs, symptoms or laboratory evidence of a viral infection or after exposure to a viral pathogen. The IRMA can be administered for prevention or for the treatment of viral infection induced CRS and other complications. The IRMA of the disclosure can administered early or late during the disease course. The IRMA of the disclosure can be administered to patients with mild, moderate or severe disease, The IRMA of the disclosure can be administered to the patients alone or in combination with other agents or treatments.

In some embodiments, the administration of the IRMA (e.g., dasatinib, ponatinib, bosutinib, tocilizumab, siltuximab etc.) or other treatment (e.g., antiviral) is at a time at which the subject does not exhibit a sign or symptom or laboratory evidence of severe cytokine release syndrome (Viral Infection Induced-viral infection induced-CRS) and/or does not exhibit grade 2 or higher Viral infection induced-CRS secondary to viral infection. In some embodiments, the administration of the IRMA (e.g., dasatinib, ponatinib, bosutinib, tocilizumab, siltuximab etc.) or other treatment (e.g., antiviral) is at a time at which the subject does not exhibit a sign or symptom of severe multi-organ toxicity. In some aspects, between the time of the initiation of the administration of the therapy and the time of the first clinical or laboratory evidence of viral infection, the subject has not exhibited severe viral infection induced-CRS and/or has not exhibited grade 2 or higher Viral infection induced-CRS.

In some embodiments, the administration of the IRMA (e.g., dasatinib, ponatinib, bosutinib, tocilizumab, siltuximab etc.) or other treatment (e.g., antiviral) is at a time at which the subject does exhibit a sign or symptom or laboratory evidence of severe cytokine release syndrome (Viral Infection Induced-viral infection induced-CRS) and/or exhibit grade 2 or higher Viral infection induced-CRS secondary to viral infection. In some embodiments, the administration of the IRMA (e.g., dasatinib, ponatinib, bosutinib, tocilizumab, siltuximab etc.) or other treatment (e.g., antiviral) is at a time at which the subject does exhibit a sign or symptom of severe multi-organ toxicity.

Provided in some embodiments are methods of treatment including administering to a subject with a viral infection or a suspected viral infection, an agent or other treatment capable of treating, preventing, delaying, or attenuating the development of complications. In some cases, the administration of the IRMA (e.g., dasatinib, ponatinib, bosutinib, tocilizumab, siltuximab etc.) or other treatment (e.g., antiviral) is at a time that is less than or no more than ten, seven, six, five, four, three, two, or one days after development of signs, symptoms or laboratory evidence of a viral infection or after exposure to a subject with a viral infection. In some embodiments, the administration of the IRMA (e.g., dasatinib, ponatinib, bosutinib, tocilizumab, siltuximab etc.) or other treatment (e.g., antiviral) is at a time at which the subject does not exhibit a sign or symptom of cytokine release syndrome (Viral infection induced-CRS) and/or does not exhibit grade 2 or higher Viral infection induced-CRS. CRS is graded according to the ASTCT CRS Consensus Grading criteria as described below.

| CRS Parameter | Grade 1 | Grade 2 | Grade 3 | Grade 4 |
|---|---|---|---|---|
| Fever [symbol] | Temperature ≥ 38° C. | Temperature ≥ 38° C. | Temperature ≥ 38° C. | Temperature ≥ 38° C. |
| | | With | | |
| Hypotension | None | Not requiring vasopressors | Requiring a vasopressor | Requiring multiple vasopressors |
| | | | with or without vasopressin | (excluding vasopressin) |
| | | And/or [symbol] | | |
| Hypoxia | None | Requiring low-flow nasal | Requiring high-flow nasal | Requiring positive pressure |
| | | cannula [symbol] or blow-by | cannula [symbol] , facemask, | (eg, CPAP, BIPAP, intubation |
| | | | nonrebreather mask, or | and mechanical ventilation) |
| | | | Venturi mask | |

[symbol]

indicates data missing or illegible when filed

Organ toxicities are graded according to the CTCAE v5.0

In some embodiments, the IRMA or other treatment (e.g., antiviral) is or comprises a steroid, or an antagonist or inhibitor of a cytokine receptor or cytokine selected from among IL-10, IL-10R, IL-6, IL-6 receptor, IFNγ, IFNGR, IL-2, IL-2R/CD25, MCP-1, CCR2, CCR4, MIPIβ, CCR5, TNF alpha, T FR1, IL-1, and IL-lRalpha/IL-lbeta. In some embodiments, the antagonist or inhibitor is or comprises an agent selected from among an antibody or antigen-binding fragment, a small molecule, a protein or peptide and a nucleic acid.

In some embodiments, the IRMA or other treatment (e.g., antiviral) is or comprises an agent selected from among tocilizumab, situximab, sarilumab, olokizumab (CDP6038), elsilimomab, ALD518/BMS-945429, sirukumab (CNTO 136), CPSI-2634, ARGX-109, FE301 and FMlOl. In some embodiments, the IRMA or other treatment (e.g., antiviral) is or comprises tocilizumab. In some embodiments, the tocilizumab is for administration in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive, or the tocilizumab is administered in a dosage amount of at least or at least about or about 2 mg/kg, 4 mg/kg, 6 mg/kg or 8 mg/kg.

In some embodiments, the agent is or comprises a steroid that optionally is or comprises a corticosteroid, which optionally is a glucocorticoid. In some embodiments, the corticosteroid is or comprises dexamethasone or prednisone. In some embodiments, the steroid is for administration in an equivalent dosage amount of from or from about 1.0 mg to 20 mg dexamethasone per day, 1.0 mg to 10 mg dexamethasone per day, or 2.0 mg to 6.0 mg dexamethasone per day, each inclusive. In some embodiments, the steroid is formulated for intravenous or oral administration.

The disclosure also provides methods or prevention of infection with viruses (e.g., coronaviruses, e.g., COVID-19, SARS virus, MERS virus, RSV, influenza, parainfluenza, by change in the local microenvironment in the oral oropharynx, nasal and nasopharynx regions. In an exemplary embodiment, the change in the microenvironment involves alkalization and/or change in the ionic composition of these regions.

### DETAILED DESCRIPTION

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “Immune response modulating agent” or “IRMA” as used herein refers to an agent that modulates the immune response. Exemplary IRMA include Tyrosine kinase inhibitors (e.g., Src kinase inhibitors, e.g., Lck inhibitors, e.g., Dasatinib, Ponatinib, Bosutinib etc.) cytokine antagonists (e.g., IL6 antagonists, Sylvant/Siltuxmab or IL6R antagonists, e.g., Tocilizumab, Sarilumab etc.), C5 inhibitor (e.g., Eculizumab) and steroids.

The term “complications of a viral infection” as described herein refers to secondary manifestations that arise as a consequent of a viral infection, such as viral pneumonia, cytokine release syndrome, shock, kidney failure, cardiac dysfunction, liver dysfunction, hematologic abnormalities etc.

The term “C5 inhibitor” as defined herein refers to an inhibitor of terminal complement 5. An exemplary C5 inhibitors includes Eculizumab.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided agents, cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, agent, cells, or composition, in the context of administration, refers to an amount effective, at dosages/amounts and for periods of time necessary, to achieve a desired result, such as a therapeutic or prophylactic result.

A “therapeutically effective amount” of an composition, e.g., a pharmaceutical formulation comprising agents or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the agents or populations of cells administered. In some embodiments, the provided methods involve administering the agents, cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

“Viral infection induced-CRS” or “Virus induced CRS) as the term used herein refers to a constellation of signs, symptoms and laboratory abnormalities related to excessive production of cytokines caused by a viral infection. A virus induced CRS is graded according to the ASTCT CRS consensus Grading criteria (Lee D W, et al, Biol Blood Marrow Transplant, 25 (2019) 625-638.

A “severe virus infection induced CRS” as the term used herein refers to a virus induced CRS of grade 2 or higher according to the ASTCT CRS consensus Grading criteria.

The term “viral infection” as used herein refers to infection with any viral pathogen. Exemplary viral infections include infection with coronavirus (e.g., SARS, MERS, SARS-cov2, etc.), infuenza A, influenza B, parainfluenza, rhinovirus, enterovirus, RSV etc.

Provided herein are methods involving early or preemptive treatment to prevent or ameliorate potential complications that may be associated with viral infections.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Methods of Preemptive Intervention with Immune Response Modulating Agents (IRMA)

In some aspects, the provided embodiments are based on observations that the complications of viral infections are caused by cytokine release. Also, in some cases, subjects with a higher cytokine release also may be at a greater risk for developing a toxicity or a more severe toxicity.

Certain available methods for treating or ameliorating complications may not always be entirely satisfactory. Many such approaches focus, for example, on targeting downstream effects of toxicity, such as by cytokine blockade, and/or delivering agents such as high-dose steroids which can also eliminate or impair the function of immune cells. Additionally, such approaches often involve administration of such interventions only upon detection of physical signs or symptoms of toxicity, which in general involve signs or symptoms of moderate or severe toxicity (e.g. moderate or severe viral infection induced-CRS or moderate or severe organ toxicity). Many of these other approaches also do not prevent other forms of toxicity such as organ toxicity, which can be associated with a viral infection.

The use of certain alternative approaches does not provide satisfactory solutions to such issues. In some cases, such agents and therapies (e.g. steroids) are themselves associated with toxic side effects.

The provided methods offer advantages over available approaches. In some embodiments, the provided methods involve the early or preemptive treatment of subjects prior to the subjects exhibiting physical signs or symptom of complications from a viral infection that are more than mild, such as prior to exhibiting physical signs or symptoms of severe complications. In some embodiments, the treatment occurs at a time in which a physical sign or symptom of mild viral infection induced-CRS, such as grade 1 viral infection induced-CRS is present, but before moderate or severe viral infection induced-CRS has developed or before grade 2 or grade 3 viral infection induced-CRS has developed. In some embodiments, the treatment occurs at a time in which a physical sign or symptom of mild organ toxicity, such as grade 1 organ toxicity is present, but before moderate or severe organ toxicity has developed or before grade 2 or grade 3 organ toxicity has developed. In some embodiments, the treatment with the IRMA occurs at a time at which no physical signs or symptom of organ toxicity has developed. Thus, in some cases, the provided methods provide the ability to intervene early before undesired end organ-related outcomes can result. In some cases, the ability to intervene early in the treatment of a toxic outcome or the potential of a toxic outcome can mean that a reduced dosage of an IRMA for treating or ameliorating the toxicity can be given and/or a decreased frequency of administration of such agent or therapy can be given.

It is to be noted, however, that the IRMA can be also administered later in the disease course. In an embodiment, an IRMA (e.g., dasatinib, ponatinib or bosutinib) and/or another treatment (e.g., antiviral) is administered to a subject exhibiting physical signs or symptoms of severe complications from a viral infection. In some embodiments, the treatment occurs at a time after moderate or severe viral infection induced-CRS has developed or before grade 2 or grade 3 viral infection induced-CRS has developed. In some embodiments, the treatment occurs at a time after moderate or severe organ toxicity has developed or after grade 2 or grade 3 organ toxicity has developed.

In an exemplary embodiment, the early administration of agents that prevent the release of cytokines or activity of cytokines, such as IL6 antagonists (e.g. Tocilizumab) or Src/Lck inhibitors (e.g., Dasatinib, Ponatinib, Bosutinib etc.) either alone or in combination to a subject with SARS-cov2 infection or COVID-19 prevents the progression of the disease and development of complications, thereby avoiding the need of ICU admission, use of vasopressors, and need of ventilatory support. In the case of a pandemic, this in turn, saves the healthcare providers from exposure to the viral pathogen, preserves the supply of personal protective equipments and ventilators and avoids the situation where the hospitals and ICU are overrun by surge of patients. Collectively, this also improves the survival of the patient in an pandemic as mortality rate in a pandemic (e.g. SARS-Cov2 pandemic) has been found to greater in hot spots where the healthcare providers, equipments and facilities are overrun by the surge of patients needing ICU admission and ventilatory support.

Exemplary such IRMA (Immune response modulating agents or toxicity-targeting agents) are described below.

In some embodiments, the IRMA (e.g. anti-cytokine agent and/or an Src/Lck antagonist e.g., Dasatinib, Ponatinib, Bosutinib etc) is administered (i) at a time that is less than or no more than ten, seven, six, five, four, three, two, one days after exposure to a viral pathogen or development of signs, symptoms or lab evidence of a viral infection. In some embodiments, the IRMA (e.g. anti-cytokine agent and/or an Src/Lck antagonist e.g., Dasatinib, Ponatinib, Bosutinib etc.) is administered at a time at which the subject does not exhibit a sign or symptom of severe cytokine release syndrome (viral infection induced-CRS) and/or does not exhibit grade 2 or higher viral infection induced-CRS. In some embodiments, the IRMA (e.g. anti-cytokine agent and/or an Src/Lck antagonist e.g., Dasatinib, Ponatinib, Bosutinib etc.) is administered at a time at which the subject exhibits a sign or symptom of severe cytokine release syndrome (viral infection induced-CRS) and/or does exhibit grade 2 or higher viral infection induced-CRS. In some embodiments, the IRMA (e.g. anti-cytokine agent and/or an Src/Lck antagonist e.g., Dasatinib, Ponatinib, Bosutinib etc.) is administered at a time at which the subject does not exhibit a sign or symptom of severe organ toxicity and/or does not exhibit grade 2 or higher organ toxicity. In some embodiments, the IRMA (e.g. anti-cytokine agent and/or an Src/Lck antagonist e.g., Dasatinib, Ponatinib, Bosutinib etc.) is administered at a time at which the subject does exhibit a sign or symptom of severe organ toxicity and/or does exhibit grade 2 or higher organ toxicity. In some embodiments, the IRMA (e.g. anti-cytokine agent and/or an Src/Lck antagonist e.g., Dasatinib, Ponatinib, Bosutinib etc.) is administered at a time at which the subject does not require supplemental oxygen. In some embodiments, the IRMA (e.g. anti-cytokine agent and/or an Src/Lck antagonist e.g., Dasatinib, Ponatinib, Bosutinib etc.) is administered at a time at which the subject requires supplemental oxygen.

In some embodiments, the provided methods are designed to or include features that result in a lower degree of complications, toxic outcome or symptom, toxicity-promoting profile, factor, or property, such as a symptom or outcome associated with or indicative of cytokine release syndrome (viral infection induced-CRS) or organ toxicity, for example, compared to administration of the therapy at a time in which the subject is not administered the IRMA or other treatment (e.g., antiviral) or is administered the IRAM or other treatment after severe viral infection induced-CRS has developed or after grade 2 or higher viral infection induced-CRS has developed. In certain embodiment, the administration of the IRMA prevents worsening of subject's condition based on 6 point Ordinal scale.

Typically, viral infection induced-CRS is caused by an exaggerated systemic immune response mediated by, for example, T cells, B cells, K cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening viral infection induced-CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure.

The incidence and timing of viral infection induced-CRS may be related to baseline cytokine levels at the time of infusion. Commonly, viral infection induced-CRS involves elevated serum levels of IL6, IL1α, IL1β, interferon (IFN)-Y, tumor necrosis factor (TNF)-a, and/or interleukin (IL)-2. Other cytokines that may be rapidly induced in viral infection induced-CRS are IL-8, and IL-10.

In some embodiments, a subject is deemed to develop “severe viral infection induced-CRS” if the subject displays: (1) fever of at least 38 degrees Celsius for at least three days; (2) cytokine elevation that includes either (a) more than 5 fold elevation of CRP and IL6 as compared to upper limit of normal reference (c) at least one clinical sign of toxicity such as hypotension (requiring at least one intravenous vasoactive pressor) or hypoxia (pO2<90%) or radiological evidence of pulmonary infection.

In some embodiments, severe viral infection induced-CRS includes viral infection induced-CRS that requires management or care in the intensive care unit (ICU).

In some embodiments, severe viral infection induced-CRS includes CRS that is between a-d on a 6 point Ordinal scale listed below.

6-point Ordinal Scale:
a. Death
b. Hospitalized, on invasive mechanical ventilation or ECMO;
c. Hospitalized, on non-invasive ventilation or high flow oxygen devices;
d. Hospitalized, requiring supplemental oxygen
e. Hospitalized, not requiring supplemental oxygen
f. Not hospitalized

In some embodiments, severe viral infection induced-CRS encompasses a combination of (1) persistent fever (fever of at least 38 degrees Celsius for at least three days) (2) persistent cough 3) a serum level of CRP of at least at or about 20 mg/dL. In some embodiments, severe viral infection induced-CRS encompasses hypotension requiring the use of two or more vasopressors or respiratory failure requiring mechanical ventilation. In some embodiments, the dosage of vasopressors is increased in a second or subsequent administration.

In some embodiments, severe viral infection induced-CRS or grade 2 or higher viral infection induced-CRS encompasses an increase in alanine aminotransferase, an increase in aspartate aminotransferase, chills, febrile neutropenia, headache, left ventricular dysfunction, renal dysfunction, hepatic dysfunction, fall in lymphocyte count, more than 3 fold elevation of CRP and/or IL6 etc.

In some embodiments, the provided methods involve early interventions prior to the development of severe viral infection induced-CRS in the subject or prior to the development of grade 2 or grade 3 viral infection induced-CRS. In some embodiments, it is understood that physical signs or symptoms of viral infection induced-CRS may exist, but such signs or symptoms are generally mild and/or are not severe. In some embodiments, the IRMA (e.g. anti-cytokine or Src/Lck antagonist agent) is administered at a time at which the subject exhibits grade 1 viral infection induced-CRS or is administered within 24 hours (e.g., within 4 hours, 6 hours, 12 hours, 16 hours etc.) after the subject exhibits a first sign or symptom of grade 1 viral infection induced-CRS. In some embodiments, the subject is administered the IRMA at a time at which a first sustained fever has developed or a time which is within 1 hours, 2 hours, 3 hours, 4 hours, 5 hours, 6 hours, 7 hours, 8 hours, 9 hours, 10 hours, 12 hours, 18 hours or 24 hours of a fever, such as a sustained fever. In some embodiments, the subject is administered the IRMA by an auto-injector. In some embodiments, the subject is self-administered the IRMA.

In some embodiments, the subject exhibits a fever, and in some aspects is treated at a time at which the subject exhibits such fever and/or exhibits or has exhibited the fever for a particular period of time.

In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the disease-targeted therapy.

In some embodiments, the subject exhibits sustained hypoxia (e.g., O2 saturation of less than 92% on room air).

In some embodiments, the IRAM is administered if the subject exhibits sustained signs, symptoms or radiological evidence of upper respiratory tract infection, such as cough, shortness of breath, chest pain, increased rate of breathing, radiological evidence of pulmonary infection (e.g., infiltrates on Chest X ray or CT scan).

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3 -hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as treatment with an antipyretic. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, one or more of the toxicity-targeting therapies (e.g. anti-cytokine therapy or Src/Lck inhibitor etc) is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

In some embodiments, the IRMA is administered prior to a physical sign or symptom of organ toxicity. In some cases, organ toxicity, including severe pulmonary toxicity or kindey toxicity or cardiac toxicity.

In some embodiments, complications of viral infection causes one more adverse events. In some embodiments, the adverse event includes, but is not limited to, an increase in alanine aminotransferase, an increase in aspartate aminotransferase, chills, febrile neutropenia, lymphopenia, hypotension, left ventricular dysfunction, kidney dysfunction, and liver dysfunction. In some embodiments, the intervention methods provided herein ameliorate or reduce such adverse events.

In some embodiments, the provided methods include administration of an IRMA at a dosage that is reduced or less than the dosage of such agent administered to a subject at a time when a physical sign or symptom of severe viral infection induced-CRS or organ toxicity has developed and/or at a time at which the subject exhibits grade 2 or grade 3 viral infection induced-CRS or organ toxicity. In some embodiments, the reduction in the dose is at least 1.2-fold, 1.3-fold, 1.4-fold, 1.5-fold, 2-fold, 3-fold, 4-fold, 5-fold, 6-fold, 7-fold, 8-fold, 9-fold or 10-fold.

### Treatment and Methods

### Immune Response Modulating Agents (IRMA) or Toxicity-Targeting Agent that Treat or Ameliorate Complications of a Viral Infection

The term “complications of a viral infection” as described herein refers to secondary manifestations that arise as a consequent of a viral infection, such as viral pneumonia, cytokine release syndrome, shock, kidney failure, cardiac dysfunction, liver dysfunction, hematologic abnormalities etc.

In some embodiments, the methods include an early or preemptive intervention or interventions, including by administration of agents or therapies or other treatments that treat a complication of a viral infection, such as viral infection induced-CRS or organ toxicity, e.g. severe viral infection induced-CRS or severe organ toxicity, and/or that prevent, delay, or attenuate the development of or risk for developing viral infection induced-CRS or organ toxicity, e.g., severe viral infection induced-CRS or severe organ toxicity. For example, in some embodiments, the agent is an Immune Response Modulating Agent (IRMA) or treatment or intervention.

In some embodiments, the IRMA, e.g., a toxicity-targeting agent, is a steroid, is an antagonist or inhibitor of a cytokine receptor, such as IL-6 receptor, CD122 receptor (IL-2Rbeta receptor), or CCR2, or is an inhibitor of a cytokine, such as IL-6, MCP-1, IL-10, IFN-γ, IL-8, or IL-18. In some embodiments, the agent is an agonist of a cytokine receptor and/or cytokine, such as TGF-β. In some embodiments, the agent, e.g., agonist, antagonist or inhibitor, is an antibody or antigen-binding fragment, a small molecule, a protein or peptide, or a nucleic acid.

In some embodiments, a fluid bolus can be employed as an intervention, such as to treat hypotension associated with viral infection induced-CRS. In some embodiments, the target hematocrit levels are >24%. In some embodiments, the intervention includes the use of absorbent resin technology with blood or plasma filtration. In some cases, the intervention includes dialysis, plasmapheresis, or similar technologies. In some embodiments, vassopressors or acetaminophen can be employed.

In some embodiments, the agent is administered at a time as described herein and in accord with the provided methods. In some embodiments, the IRMA is administered at a time that is within, such as less than or no more than, 1, 3, 4, 5, 6, 7, 8, 9 or 10 days after development of first sign or symptom or lab evidence of viral infection or exposure to viral pathogen. In some embodiments, the toxicity-targeting agent is administered within or within about 1 day, 2 days or 3 days after development of first sign or symptom or lab evidence of viral infection or exposure to viral pathogen.

In some embodiments, the IRMA, e.g., toxicity-targeting agent, is administered to a subject after development of first sign or symptom or lab evidence of viral infection or exposure to viral pathogen at a time at which the subject does not exhibit grade 2 or higher viral infection induced-CRS or grade 2 or higher organ toxicity. In some aspects, the IRMA is administered after development of first sign or symptom or lab evidence of viral infection or exposure to viral pathogen at a time at which the subject does not exhibit severe viral infection induced-CRS or severe organ toxicity.

Non limiting examples of symptoms of viral infection include, cough, fever, running nose, shortness of breath, headache, chest pain, diarrhea, nausea, vomiting, malaise etc.

Non limiting examples of lab abnormalities of viral infection include elevation of CRP, IL6, ferritin, D-dimers, LDH, creatinine, blood urea nitrogen, AST, ALT and the like.

Non limiting examples of viral infections include coronaviruses (e.g. SARS-Cov2 (COVID-19)), Influenza A, Influenza B, Parainfluenza, RSV, enterovirus and the like.

Non-limiting examples of interventions for treating or ameliorating a complication of a viral infection, such as severe viral infection induced-CRS (viral infection induced-CRS), include dasatinib, tocilizumab or other IRMA as described, which can be at a time in which there is a sustained or persistent fever of greater than or about 38° C. or greater than or greater than about 39° C. in the subject. In some embodiments, the fever is sustained in the subject for more than 4 hours, more than 12 hours, more than 16 hours, or more than 24 hours before intervention.

Non-limiting examples of interventions for treating or ameliorating a toxicity, such as severe viral infection induced-CRS, include dasatinib, tocilizumab or other IRMA as described herein, which can be at a time in which there is a sustained or persistent cough of greater than or about 38° C. or greater than or greater than about 39° C. in the subject. In some embodiments, the fever is sustained in the subject for more than 4 hours, more than 12 hours, more than 16 hours, or more than 24 hours before intervention.

In some cases, the agent or therapy or intervention, e.g., IRMA, is administered alone or is administered as part of a composition or formulation, such as a pharmaceutical composition or formulation, as described herein. Thus, the agent alone or as part of a pharmaceutical composition can be administered intravenously or orally, or by any other acceptable known route of administration or as described herein.

In some embodiments, the dosage of agent or the frequency of administration of the agent in a dosage regimen is reduced compared to the dosage of the agent or its frequency in a method in which a subject is treated with the agent after grade 2 or higher viral infection induced-CRS or organ toxicity, such as after severe, e.g., grade 3 or higher, viral infection induced-CRS or after severe, e.g., grade 3 or higher organ toxicity, has developed or been diagnosed (e.g. after physical signs or symptoms of grade 3 or higher viral infection induced-CRS or organ toxicity has manifested).

### Agents Targeting Viral Induced Complications (i.e. Toxicity Targeting Agents)

### Tyrosine Kinase Inhibitors

In some embodiments, the agent, e.g., toxicity-targeting agent, that treats, prevents, delays, or attenuates the development of complications from a viral infection (e.g., COVID-19), such as grade 1 or higher or viral infection induced-CRS or organ toxicity, is a Tyrosine kinase inhibitor (TKI) (e.g., a Src kinase inhibitor).

In some embodiments, the agent, e.g., toxicity-targeting agent, that treats, prevents, delays, or attenuates the development of complications from a viral infection (e.g., COVID-19), such as grade 1 or higher or viral infection induced-CRS or organ toxicity, is a Lck inhibitor. Exemplary Lck inhibitors include Dasatinib, Ponatinib, Bosutinib.

In some embodiments, the agent is a Tyrosine kinse inhibitor (e.g., a Src inhibitor, e.g. a Lck inhibitor, e.g., dasatinib, ponatinib or Bosutinib etc.) and is administered in an amount that is therapeutically effective to prevent, treat, ameliorate or reduce one or more signs, symptoms, lab abnormalities of a complication due to a viral infection, such as viral infection induced-CRS or organ toxicity. In some embodiments, indicators of improvement or successful treatment include determination of the failure to manifest a relevant score on toxicity grading scale (e.g. viral infection induced-CRS or organ toxicity grading scale), such as a score of less than 3, or a change in grading or severity on the grading scale as discussed herein, such as a change from a score of 4 to a score of 3, or a change from a score of 4 to a score of 2, 1 or 0. In some embodiments, indicators of improvement or successful treatment include improvements in one or more of the clinical parameters, such as need for supplemental oxygen, vasopressors, ventilatory support or ICU stay. In some embodiments, indicators of improvement or successful treatment include improvement in one or more of the laboratory parameters, such as serum level of CRP, IL6, LDH, serum creatinine, blood urea nitrogen, AST, ALT, and lymphocyte count etc.

In some aspects, the Tyrosine kinse inhibitor (e.g., a Src inhibitor, e.g. a Lck inhibitor, e.g., dasatinib, ponatinib or Bosutinib etc.) is provided in a therapeutically effective dose. Therapeutically effective concentration can be determined empirically by testing in known in vitro or in vivo (e.g. animal model) systems. For example, the amount of a selected Tyrosine kinse inhibitor (e.g., a Src inhibitor, e.g. a Lck inhibitor, e.g., dasatinib, ponatinib or Bosutinib etc.) to be administered to ameliorate complications, such as viral infection induced-CRS or organ toxicity, can be determined by standard clinical techniques. In addition, animal models can be employed to help identify optimal dosage ranges. The precise dosage, which can be determined empirically, can depend on the particular therapeutic preparation, the regime and dosing schedule, the route of administration and the seriousness of the disease.

The TKI (e.g., a Src inhibitor, e.g. a Lck inhibitor, e.g., dasatinib, ponatinib or Bosutinib etc.) can be administered in any amount that is effective to ameliorate one or more symptoms associated with the viral infection, such as with the viral infection induced-CRS or organ toxicity. The TKI (e.g., a Src inhibitor, e.g. a Lck inhibitor, e.g., dasatinib, ponatinib or Bosutinib etc.), can be administered, for example, at an amount between at or about 0.1 and 200 mg, per dose, 0.1 to 100 mg, 0.1 to 80 mg, 0.1 to 40 mg, 0.1 to 30 mg, 0.1 to 20 mg, 0.1 to 15 mg, 0.1 to 10 mg, 0.1 to 5 mg, 0.2 to 40 mg, 0.2 to 30 mg, 0.2 to 20 mg, 0.2 to 15 mg, 0.2 to 10 mg, 0.2 to 5 mg, 0.4 to 40 mg, 0.4 to 30 mg, 0.4 to 20 mg, 0.4 to 15 mg, 0.4 to 10 mg, 0.4 to 5 mg, 0.4 to 4 mg, 1 to 20 mg, 1 to 15 mg or 1 to 10 mg, to a 70 kg adult human subject. Typically, the TKI (e.g., a Src inhibitor, e.g. a Lck inhibitor, e.g., dasatinib, ponatinib or Bosutinib etc.) is administered at an amount between at or about 5 and 200 mg, for example, at or about 5 mg, 10 mg, 20 mg, 30 mg, 45 mg, 50 mg, 60 mg, 80 mg, 100 mg, 150 mg, 180 mg, 200 mg per dose, to an average adult human subject. The TKI can be administered at different frequency, such as once daily, twice daily, three times daily or four times daily etc.

In some embodiments, the TKI (e.g., a Src inhibitor, e.g. a Lck inhibitor, e.g., dasatinib, ponatinib or Bosutinib etc.) can be administered, for example, at a dosage of at or about 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 2 mg/kg, 2.5 mg/kg or 3 mg/kg, to an average adult human subject, typically weighing about 70 kg to 75 kg.

The TKI (e.g., a Src inhibitor, e.g. a Lck inhibitor, e.g., dasatinib, ponatinib or Bosutinib etc.) can be administered orally (tablets, liquid or liquid concentrate), PO, intravenously (IV), intramuscularly or by any other known route or route described herein (e.g., with respect to pharmaceutical formulations). In some aspects, the TKI is administered as a bolus, and in other aspects it may be administered over a period of time.

In some aspects, the TKI (e.g., a Src inhibitor, e.g. a Lck inhibitor, e.g., dasatinib, ponatinib or Bosutinib etc.) can be administered over a period of more than one day, such as over two days, over 3 days, or over 4, over 7 days, over 10 days, over 15 days, over 21 days, over 30 days or more days. In some embodiments, the TKI can be administered one per day, twice per day, or three times or more per day.

In some embodiments, the dosage of TKI (e.g., a Src inhibitor, e.g. a Lck inhibitor, e.g., dasatinib, ponatinib or Bosutinib etc.), is administered in successively lower dosages per treatment. Hence, in some such treatment regimes, the dose of TKI (e.g., a Src inhibitor, e.g. a Lck inhibitor, e.g., dasatinib, ponatinib or Bosutinib etc.) is tapered.

Generally, the dose of TKI (e.g., a Src inhibitor, e.g. a Lck inhibitor, e.g., dasatinib, ponatinib or Bosutinib etc.) administered is dependent upon the specific TKI, as a difference in potency exists between different TKI. It is typically understood that drugs vary in potency, and that doses can therefore vary, in order to obtain equivalent effects.

In some embodiments, the TKI (e.g., a Src inhibitor, e.g. a Lck inhibitor, e.g., dasatinib, ponatinib or Bosutinib etc.) is administered if fever persists for more than 4 hours in a patient with a viral infection, suspected viral infection or exposure to a viral infection. In an embodiment, the virus is SARS-cov2 and virus infection is COVID-19. In an embodiment, the TKI is administered before, after or concomitantly with treatment with an IL6 or IL6R antagonist, e.g., tocilizumab or siltuximab. In some embodiments, TKI is administer before, after or concomitantly with treatment with steroids. In some embodiment, TKI is administered concurrently with steroids and/or an IL6 or IL6R antagonist (e.g., tocilizumab or Sylvant). In some embodiments, TKI is administered concurrently with or subsequent to oxygen supplementation. In some embodiments, TKI is administered concurrently with or subsequent to C5 inhibitors (Eculizumab). In some embodiments, TKI is administered concurrently with or subsequent to vasopressors. In some embodiments, TKI is administer before, after or concomitantly with treatment with other agents, such as antiviral drugs (e.g. Remdesivir, Lopinavir/Ritonavir, and Favilavir), e.g., hydroxychloroquine, chloroquine, and Azithromycin. In an embodiment, the IL6/IL6R antagonists, C5 inhibitors steroids, antiviral and other agents are used at their standard doses when combined with the TKIs.

The method involves administration to a subject with a viral infection a tyrosine kinase inhibitor. In a preferred embodiment, the tyrosine kinase inhibitor is a Src kinase inhibitor. In a more preferred embodiment, the Src kinase inhibitor is a Lck inhibitor. In a more preferred embodiment, the tyrosine kinase inhibitor is dasatinib, saracatinib, bosutinib, nilotinib, or PPl-inhibitor. In a more preferred embodiment, the inhibitor is bosutinib. In a more preferred embodiment, the inhibitor is saracatinib. in a more preferred embodiment, the inhibitor is nilotinib. in a more preferred embodiment, the inhibitor is PPl-inhibitor. In an even more preferred embodiment, the inhibitor is dasatinib.

In one embodiment, dasatinib is administered orally at a dose of at least 10 mg/day, 20 mg/day, 40 mg/day, 60 mg/day, 70 mg/day, 90 mg/day, 100 mg/day, 140 mg/day, 180 mg/day, 210 mg/day, 250 mg/day or 280 mg/day. In an embodiment, Dasatinib is administered in split doses. e.g., 70 mg orally twice a day. In an embodiment, Dasatinib is administered with food.

In an embodiment, dasatinib is to be administered continuously such that the serum levels of dasatinib are continuously at or above 50 nM. In another preferred embodiment, dasatinib is to be administered continously, such that the serum levels of dasatinib are not continuously at or above 10 nM. In an exemplary embodiment, dasatinib is to be administered daily, wherein the daily administration comprises oral administration of 50-200 mg dasatinib daily, preferably 100 mg daily.

In one embodiment, ponatinib is administered orally at a dose of at least 15 mg/day, 30 mg/day, 45 mg/day, 60 mg/day. In an embodiment, ponatinib is administered in split doses; e.g., 15 mg orally twice a day.

In an embodiment, Bosutinib is administered orally at a dose of 100 mg to 600 mg/day. In an embodiment, Bosutinib is administered in split doses; e.g., 200 mg orally twice a day.

In another embodiment, the subject is further administered one or more IRMA (immune response modulating agent) to manage elevated levels of a soluble factor produced during viral infection. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an IRMA administered to prevent complications of viral infection can be an agent that neutralizes one or more of these soluble factors. In one embodiment, the IRMA that neutralizes one or more of these soluble forms is small molecule, an antibody or antigen binding fragment thereof. Examples of such agents include, but are not limited to a steroid (e.g., corticosteroid), an inhibitor of TNFα or TNF receptor 1 or TNF receptor 2, an inhibitor of IL-6 or IL6R, an inhibitor of C5, or an inhibitor of TRAIL signaling (e.g., an antibody against TRAIL, DR4-Fc or DR5-Fc). An example of a TNFa inhibitor is an anti-TNFa antibody molecule such as, infliximab, adalimumab, certolizumab pegol, and golimumab. Another example of a TNFa inhibitor is a fusion protein such as entanercept. An example of an IL-6 inhibitor is an anti-IL-6 antibody molecule or an anti-IL-6 receptor antibody molecule such as tocilizumab, sarilumab, elsilimomab, CNTO 328, ALD518/BMS-945429, CNTO 136, CPSI-2364, CDP6038, VX30, ARGX-109, FE301, and FM101.

In an embodiment, the IRMA is administered by oral, intravenous, intramuscular, intradermal, intranasal, intraperitoneal or subcutaneous route. In an embodiment, the IRAM (e.g., Tocilizumab, Sarilumab etc.) is administered by an autoinjector. In an embodiment, the IRMA inhibitor (e.g., Tocilizumab, Sarilumab etc.) is self-administered.

In an embodiment, the IRMA is an IL6R inhibitor Tocilizumab, Sarilumab etc.) and IL6R inhibitor is administered by intravenous, intramuscular, intradermal, intranasal, intraperitoneal or subcutaneous route. In an embodiment, the IL6R inhibitor (e.g., Tocilizumab, Sarilumab etc.) is administered by an autoinjector. In an embodiment, the IL6R inhibitor e.g., Tocilizumab, Sarilumab etc.) is self-administered.

In one embodiment, the anti-IL-6 receptor antibody molecule is tocilizumab. In some embodiments, the one or more doses of tocilizumab is administered intravenously to the subject at a dose of about 8 mg/kg. In an embodiment, Tocilizumab is administered by subcutaneous route. In an embodiment, Tocilizumab is administered by intramuscular route. In an embodiment, Tocilizumab is administered by an autoinjector. In an embodiment, Tocilizumab is self-administered.

In one embodiment, the anti-IL-6 eceptor antibody molecule is Sarilumab. In an embodiment, the one or more doses of Sarilumab is administered intravenously to the subject at a dose of about 200 mg every 2 weeks. In an embodiment, Sarilumab is administered by subcutaneous route. In an embodiment, Sarilumab is administered by intramuscular route. In an embodiment, Sarilumab is administered by an autoinjector. In an embodiment, Sarilumab is self-administered.

In one embodiment, the anti-I 6 antibody molecule is Sylvant (Siltuximab). In an embodiment, the one or more doses of Siltuximab is administered intravenously to the subject at a dose of 11 mg/kg. In an embodiment, Siltuximab is administered by subcutaneous route. In an embodiment, Siltuximab is administered by intramuscular route. In an embodiment, Siltuximab is administered by an autoinjector. In an embodiment, Siltuximab is self-administered.

In some embodiments, the method further comprises administering to the subject an effective amount of a corticosteroid. In some embodiments, the corticosteroid is administered intravenously to the subject. In some embodiments, the corticosteroid is methylprednisolone. In some embodiments, the methylprednisolone is administered at a dose of about 1-2 mg/kg per day. In an exemplary embodiment, the steroid is Methylprednisolone and the subject is administered Methylprednisolone at a dose of 500 mg IV every 12 hours for 3 days followed by followed by 250 mg IV every 12 hours for 2 days, 125 mg IV every 12 hours for 2 days, 60 mg IV every 12 hours until virus-induced CRS improvement to Grade 1 and then taper over 2 weeks. In other embodiments, the corticosteroid is dexamethasone. In some embodiments, the dexamethasone is administered at a dose of about 10 mg every 12 hours until stabilization of subject's hemodynamic parameters and then tapered over 1-2 weeks.

In an embodiment, the alternate regimen consists of Tocilizumab and Tocilizumab is administered at a dose of 4-8 mg/kg IV for up to 3 doses in a 24 hour period and maximum 4 doses. In an embodiment, the alternate regimen consists of siltuxiamab and siltuximab is administered at a dose of 5-11 mg/kg IV once or 11 mg/Kg subcutaneously.

In one embodiment, the IL-6 inhibitor is a camelid bispecific antibody that binds to IL6R and human serum albumin (e.g., IL6R-304-Alb8).

An example of a IL1 based inhibitor is anakinra. In an embodiment, the subject is also administered a C5 inhibitor (e.g., tesidolumab, or eculizumab)

In some embodiments, the subject with a viral infection is administered a Tyrosine kinase inhibitor (e.g., a Src inhibitor, e.g., a Lck inhibitor, e.g., Dasatinib) either alone or in combination with other agents (e.g., Tocilizumab, siltuxiamab, anakinra tesidolumab, or eculizumab etc.) if the subject manifests one or more symptom and signs selected from among persistent fever greater than at or about 38 degrees Celsius; hypotension (systolic blood pressure of <90 mm Hg and/or diastolic blood pressure of <60 mm Hg); hypotension requiring high dose vasopressor or multiple vasopressors; hypotension that is unresponsive to intravenous fluids; hypoxia, which optionally comprises (e.g., plasma oxygen (pO2) levels of less than at or about 90%; Imaging studies (e.g., X-Ray or CT-scan) consistent with pulmonary infiltrate, and respiratory failure requiring mechanical ventilation.

A variety of additional tests can be performed on a subject to determine whether the subject has increased inflammatory cytokines and is a candidate for Tyrosine kinase inhibitor, inhibitor of cytokines (e.g., Tocilizumab, siltuxiamab, anakinra tesidolumab, or eculizumab etc.) and/or combination. These tests include one or more of the following
Elevated level of serum C-reactive protein (CRP) (i.e. CRP>1.5 times the upper limit of reference range, or CRP>20 mg/L or doubling of CRP within the last 24 hours)
Elevated level of serum Interleukin 6 (IL6) (i.e. IL6>1.5 times the upper limit of reference range, or IL6>5 pg/mL or doubling of IL6 within the last 24 hours)
Elevated lactate dehydrogenase (any elevation above normal range);
Elevation of serum ferritin
Lymphopenia
Elevation of D-dimers
Thrombocytopenia with platelet count <50x10e9/L;
Anemia below lower limit of normal or anemia requiring transfusion support as per center standard;
Schistocytes on peripheral blood smear (>2 per HPF) or histologic evidence of Microangiopathy; and/or
Low Haptoglobin level.

In some embodiments, the subject is administered one or more IRMA selected from the group of a Tyrosine kinase inhibitor (e.g., a Src inhibitor, e.g., a Lck inhibitor, e.g., Dasatinib), IL6R antagonist (e.g., Tocilizumab, sarilumab etc.), IL6 antibody (e.g., siltuxiamab), IL1 antagonist (e.g., anakinra), C5 inhibitor (e.g. tesidolumab, or eculizumab etc.), and steroids, either alone or in combination, if the subject shows elevation of serum C-reactive protein (CRP), serum IL6, serum IL1α, IL1β, or D-dimers. In an embodiment, the subject is administered one or more IRMA selected from the group of a Tyrosine kinase inhibitor (e.g., a Src inhibitor, e.g., a Lck inhibitor, e.g., Dasatinib), IL6R antagonist (e.g., Tocilizumab, sarilumab etc.), IL6 antibody (e.g., siltuxiamab), IL1 antagonist (e.g. anakinra), C5 inhibitor tesidolumab, or eculizumab etc.), and steroids, either alone or in combination, if the serum CRP, serum IL6, serum IL1α, IL1β, or D-dimers are elevated more than 1.5 fold (e.g., greater than 1.75 fold, 2 fold, 2.5 fold, 3 fold, 5 fold) the upper limit of normal. In an embodiment, the subject is administered one or more IRMA selected from the group of a Tyrosine kinase inhibitor (e.g., a Src inhibitor, e.g., a Lck inhibitor, e.g., Dasatinib), IL6R antagonist (e.g., Tocilizumab, sarilumab etc.), IL6 antibody (e.g., siltuxiamab), IL1 antagonist (e.g. anakinra), C5 inhibitor (e.g. tesidolumab, or eculizumab etc.), and steroids, either alone or in combination, until the serum CRP, serum IL6, serum IL1α, IL1β, or D-dimers normalize.

In some embodiments, if the subject's hemodynamic parameters and oxygenation status does not resolve or worsens within 24 hours of treating the signs and symptoms of the virus-induced CRS event, the method further comprises administering to the subject one or more additional doses of a IL6R antagonist (e.g., tocilizumab), IL6 antagonis (e.g. siltuximab), IL1 antagonist (e.g., anakinra), C5 inhibitor (e.g. tesidolumab, or eculizumab) to manage virus-induced CRS.

The disclosure also provides a method to identify subjects at high risk of developing complications from respiratory tract infection (e.g. COVID-19, SARS, MERS, coronavirus, Influenza virus, RSV, parainfluenza virus etc.) based on monitoring the level of serum markers. In an embodiment, the serum markers include serum CRP, serum IL6, serum IL1α, serum IL1β, serum ferritin, or D-dimers.

### Steroid

In some embodiments, the IRMA, e.g., toxicity-targeting agent, that treats, prevents, delays, or attenuates the development of complications from a viral infection (e.g., COVID-19), such as grade 1 or higher or viral infection induced-CRS or organ toxicity, is a steroid, e.g., corticosteroid. Corticosteroids typically include glucocorticoids and mineralocorticoids.

Any corticosteroid, e.g., glucocorticoid, can be used in the methods provided herein. In some embodiments, glucocorticoids include synthetic and non-synthetic glucocorticoids.

In some examples, the glucocorticoid is selected from among cortisones, dexamethasones, hydrocortisones, methylprednisolones, prednisolones and prednisones. In a particular example, the glucocorticoid is dexamethasone.

In some embodiments, the IRMA is a corticosteroid and is administered in an amount that is therapeutically effective to prevent, treat, ameliorate or reduce one or more symptoms of a complication due to a viral infection, such as viral infection induced-CRS or organ toxicity. In some embodiments, indicators of improvement or successful treatment include determination of the failure to manifest a relevant score on toxicity grading scale (e.g. viral infection induced-CRS or organ toxicity grading scale), such as a score of less than 3, or a change in grading or severity on the grading scale as discussed herein, such as a change from a score of 4 to a score of 3, or a change from a score of 4 to a score of 2, 1 or 0. In some embodiments, indicators of improvement or successful treatment include improvement in one or more of the clinical parameters, such as need for supplemental oxygen, vasopressors, ventilatory support or ICU stay. In some embodiments, indicators of improvement or successful treatment include improvement in one or more of the laboratory parameters, such as serum level of CRP, IL6, LDH, serum creatinine, blood urea nitrogen, AST, ALT, and lymphocyte count etc.

In some aspects, the corticosteroid is provided in a therapeutically effective dose. Therapeutically effective concentration can be determined empirically by testing in known in vitro or in vivo (e.g. animal model) systems. For example, the amount of a selected corticosteroid to be administered to ameliorate complications, such as viral infection induced-CRS or organ toxicity, can be determined by standard clinical techniques. In addition, animal models can be employed to help identify optimal dosage ranges. The precise dosage, which can be determined empirically, can depend on the particular therapeutic preparation, the regime and dosing schedule, the route of administration and the seriousness of the disease.

The corticosteroid can be administered in any amount that is effective to ameliorate one or more symptoms associated with the viral infection, such as with the viral infection induced-CRS or organ toxicity. The corticosteroid, e.g., glucocorticoid, can be administered, for example, at an amount between at or about 0.1 and 100 mg, per dose, 0.1 to 80 mg, 0.1 to 60 mg, 0.1 to 40 mg, 0.1 to 30 mg, 0.1 to 20 mg, 0.1 to 15 mg, 0.1 to 10 mg, 0.1 to 5 mg, 0.2 to 40 mg, 0.2 to 30 mg, 0.2 to 20 mg, 0.2 to 15 mg, 0.2 to 10 mg, 0.2 to 5 mg, 0.4 to 40 mg, 0.4 to 30 mg, 0.4 to 20 mg, 0.4 to 15 mg, 0.4 to 10 mg, 0.4 to 5 mg, 0.4 to 4 mg, 1 to 20 mg, 1 to 15 mg or 1 to 10 mg, to a 70 kg adult human subject.

In some embodiments, the corticosteroid can be administered, for example, at a dosage of at or about 0.001 mg/kg (of the subject), 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, 0.02 mg/kg, 0.025 mg/kg, 0.03 mg/kg, 0.035 mg/kg, 0.04 mg/kg, 0.045 mg/kg, 0.05 mg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

The corticosteroid, or glucocorticoid, for example dexamethasone, can be administered orally (tablets, liquid or liquid concentrate), PO, intravenously (IV), intramuscularly or by any other known route or route described herein (e.g., with respect to pharmaceutical formulations). In some aspects, the corticosteroid is administered as a bolus, and in other aspects it may be administered over a period of time.

In some aspects, the glucocorticoid can be administered over a period of more than one day, such as over two days, over 3 days, or over 4 or more days. In some embodiments, the corticosteroid can be administered one per day, twice per day, or three times or more per day. For example, the corticosteroid, e.g., dexamethasone, may in some examples be administered at 10 mg (or equivalent) IV twice a day for three days.

In some embodiments, the dosage of corticosteroid, e.g., glucocorticoid, is administered in successively lower dosages per treatment.

Generally, the dose of corticosteroid administered is dependent upon the specific corticosteroid, as a difference in potency exists between different corticosteroids.

Thus, in some embodiments, the steroid is administered in an equivalent dosage amount of from or from about 1.0 mg to 20 mg dexamethasone per day.

### Cytokine (e.g., IL6) and Cytokine Receptor (IL6R) Antagonists

In some embodiments, the IRMA, e.g. toxicity-targeting agent, that prevents, treats or ameliorates signs, symptoms or complications of a viral infection (e.g., COVID-19), such as viral infection induced-CRS or organ toxicity, is one that targets a cytokine, e.g., is an antagonist or inhibitor of a cytokine, such as transforming growth factor beta (TGF-beta), interleukin 6 (IL-6), interleukin 10 (IL-10), IL-2, MIPIβ (CCL4), TNF alpha, IL-1, interferon gamma (IFN-gamma), or monocyte chemoattractant protein-1 (MCP-1). In some embodiments, the IRMA that treats or ameliorates symptoms of a toxicity of an immunotherapy and/or a cell therapy, such as viral infection induced-CRS or organ toxicity, is one that targets (e.g. inhibits or is an antagonist of) a cytokine receptor, such as IL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIPIβ receptor (e.g., CCR5), TNF alpha receptor (e.g., TNFRl), IL-1 receptor (ILl-Ra/IL-lRp), or IL-10 receptor (IL-1 OR).

The amount of a selected IRMA that prevents, treats or ameliorates signs, symptoms, laboratory abnormalities or complications of a viral, such as viral infection induced-CRS or organ toxicity can be determined by standard clinical techniques. Exemplary signs, symptoms, lab abnormalities or complications include, but are not limited to, an increase in serum CRP, IL6, LDH, alanine aminotransferase (ALT), serum creatinine, blood urea nitrogen, aspartate aminotransferase (AST), chills, fever, shortness of breath, hypoxia, tachycardia, hypotension, lymphopenia, radiological evidence of pulmonary infiltrates (e.g. pneumonia).

In some embodiments, the IRMA is administered in a dosage amount of from or from about 30 mg to 5000 mg.

In some embodiments, the IRMA is administered from or from about 0.5 mg/kg to 100 mg/kg, such as from or from about 1 mg/kg to 50 mg/kg, 1 mg/kg to 25 mg/kg, 1 mg/kg to 10 mg/kg, 1 mg/kg to 5 mg/kg, 5 mg/kg to 100 mg/kg, 5 mg/kg to 50 mg/kg, 5 mg/kg to 25 mg/kg, 5 mg/kg to 10 mg/kg, 10 mg/kg to 100 mg/kg, 10 mg/kg to 50 mg/kg, 10 mg/kg to 25 mg/kg, 25 mg/kg to 100 mg/kg, 25 mg/kg to 50 mg/kg to 50 mg/kg to 100 mg/kg. In some embodiments, the agent is administered at a dose of 4 mg/kg or 8 mg/kg.

In some embodiments, the IRMA is administered by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery.

In some embodiments, the amount of the IRMA (immune response modulating agent) is administered about or approximately twice daily, daily, every other day, three times a week, weekly, every other week or once a month.

In some embodiments, the IRMA is tocilizumab. In some embodiments, tocilizumab is administered as an early intervention in accord with the provided methods a dosage of from or from about 1 mg/kg to 12 mg/kg, such as at or about 4 mg/kg, 8 mg/kg, or 10 mg/kg. In some embodiments, tocilizumab is administered by intravenous infusion. In some embodiments, tocilizumab is administered by intramuscular, subcutaneous, intradermal, or intraperitoneal routes. In some embodiments, tocilizumab is administered by autoinjector. In some embodiments, tocilizumab is self-administered. In some embodiments, tocilizumab is administered for a persistent fever of greater than 38.4° C. (e.g., greater than 39.0° C., 39.5° C., 40.0° C. etc) lasting 4 hours that is unresponsive to acetaminophen. In some embodiments, a second administration of tocilizumab is provided if symptoms recur after 48 hours of the initial dose.

In one embodiment, the IRMA is Sarilumab. In an embodiment, the one or more doses of Sarilumab is administered subcutenously to the subject at a dose of about 200 mg every 2 weeks. In an embodiment, Sarilumab is administered by intramuscular route. In an embodiment, Sarilumab is administered by an autoinjector. In an embodiment, Sarilumab is self-administered.

In one embodiment, the IRMA is Sylvant (Siltuximab). In an embodiment, the one or more doses of Siltuximab is administered intravenously to the subject at a dose of 11 mg/kg. In an embodiment, Siltuximab is administered by subcutaneous route. In an embodiment, Siltuximab is administered by intramuscular route. In an embodiment, Siltuximab is administered by an autoinjector. In an embodiment, Siltuximab is self-administered.

In some embodiments, the agent is an agonist or stimulator of TGF-β or a TGF-β receptor (e.g., TGF-β receptor I, II, or III). In some aspects, the agent is an antibody that increases TGF-β activity, such as an antibody or antigen-binding fragment that binds to TGF-β or one of its receptors. In some embodiments, the agent that is an agonist or stimulator of TGF-β and/or its receptor is a small molecule, a protein or peptide, or a nucleic acid.

In some embodiments, the agent is an antagonist or inhibitor of MCP-1 (CCL2) or a MCP-1 receptor (e.g., MCP-1 receptor CCR2 or CCR4). In some aspects, the agent is an antibody that neutralizes MCP-1 activity, such as an antibody or antigen-binding fragment that binds to MCP-1 or one of its receptors (CCR2 or CCR4). In some embodiments, the MCP-1 antagonist or inhibitor is any described in Gong et al. J Exp Med. 1997 Jul. 7; 186(1): 131-137 or Shahrara et al. J Immunol 2008; 180:3447-3456. In some embodiments, the agent that is an antagonist or inhibitor of MCP-1 and/or its receptor (CCR2 or CCR4) is a small molecule, a protein or peptide, or a nucleic acid.

In some embodiments, the agent is an antagonist or inhibitor of IFN-γ or an IFN-γ receptor (IFNGR). In some aspects, the agent is an antibody that neutralizes IFN-γ activity, such as an antibody or antigen-binding fragment that binds to IFN-γ or its receptor (IFNGR). In some aspects, the IFN-gamma neutralizing antibody is any described in Dobber et al. Cell Immunol. 1995 February; 160(2): 185-92 or Ozmen et al. J Immunol. 1993 Apr. 1; 150(7):2698-705. In some embodiments, the agent that is an antagonist or inhibitor of IFN-y/IFNGR is a small molecule, a protein or peptide, or a nucleic acid.

In some embodiments, the agent is an antagonist or inhibitor of IL-10 or the IL-10 receptor (IL-IOR). In some aspects, the agent is an antibody that neutralizes IL-10 activity, such as an antibody or antigen-binding fragment that binds to IL-10 or IL-10R. In some aspects, the IL-10 neutralizing antibody is any described in Dobber et al. Cell Immunol. 1995 February; 160(2): 185 -92 or Hunter et al. J Immunol. 2005 Jun. 1; 174(11):7368-75. In some embodiments, the agent that is an antagonist or inhibitor of IL-10/IL-lOR is a small molecule, a protein or peptide, or a nucleic acid.

### C5 Inhibitors

In another aspect, the disclosure provides a method for use of a drug, e.g. antibody, capable of inhibiting the complement pathway, e.g. an anti-C5 antibody, for the prevention and treatment of complications associated with a viral infection.

In an embodiment, the viral infection is caused by a coronavirus (SARS-cov2, SARS, MERS etc.), influenza A, influenza B, parainfluenza, RSV, Rhinovirus, Enterovirus and the like.

In a certain aspect, the drug may comprise a complement inhibitor, e.g. Coversin or an antibody capable of inhibiting the complement pathway, e.g. an anti-C5 antibody capable of inhibiting the complement pathway. In one aspect, the antibody may comprise a monoclonal antibody capable of inhibiting the complement pathway. In other aspects, the drug may comprise a human monoclonal antibody or a humanized monoclonal antibody capable of inhibiting the complement pathway, e.g. a human monoclonal or humanized monoclonal anti-C5 antibody capable of inhibiting the complement pathway.

In certain embodiments, a complement inhibitor may be an antibody capable of inhibiting complement, such as an antibody that can block the formation of the membrane attack complex (MAC). For example, an antibody complement inhibitor may include an antibody that binds C5. Such anti-C5 antibodies may directly interact with C5 and/or C5b, so as to inhibit the formation of and/or physiologic function of C5b.

Suitable anti-C5 antibodies are known to those of skill in the art. Antibodies can be made to individual components of activated complement, e.g., antibodies to C7, C9, etc. (see, e.g., U.S. Pat. No. 6,534,058; US patent application US 20030129187; and U.S. Pat. No. 5,660,825). WO2010015608 and W0199529697 teach antibodies which binds to C5 and inhibit cleavage into C5a and C5b thereby decreasing the formation not only of C5a but also the downstream complement components.

### JAK1/2 Inhibitors

In an embodiment, the IRMA of the disclosure is administered in combination with and JAK inhibitor. In an embodiment, the JAK inhibitor is an inhibitor of JAK1, JAK2, JAK3 or a combination of different JAK. In an embodiment, the JAK inhibitor is tofacitinib, baricitinib and upadacitinib.

### Antiviral Agents

In an embodiment, the IRMA of the disclosure is administered in combination with an antiviral agent. In a non-limiting embodiment, the antiviral agent is any one or more of the Chloroquine, hydroxychloroquine (HC), lopinavir/ritonavir (LPV/r, Kaletra), remdesivir, Oseltamivir Phosphate, ribavarine, Favilavir and XOFLUZA™ (baloxavir marboxil).

In an embodiment, Chloroqine is used at a dose of between about 200 mg to 1 g per day. In an embodiment, hydroxy-chloroqine is used at a dose of between about 200 mg to 600 mg per day. In an embodiment, lopinavir/ritonavir (LPV/r, Kaletra) is used at a dose of around 200 mg/50 mg PO 4 times a day orally. In an embodiment, lopinavir/ritonavir (LPV/r, Kaletra) is used at a dose of around 100 mg/25 mg PO 4 times a day orally. In an embodiment, remdesivir is used at a dose of between 100 to 300 mg per day. In an embodiment, remdesivir is used at a dose of a 200 mg dose of the drug on day one, followed by a 100 mg dose every day until day five or ten. In an embodiment, Oseltamivir Phosphate is used at dose of between 50 to 250 mg per day.

In an embodiment, Oseltamivir Phosphate is used at dose of 75 mg per day. In an embodiment, Favilavir is used at dose of between 50 to 250 mg per day.

### Other Agents

In an embodiment, the IRMA of the disclosure is administered in combination with Azithromycin.

### Compositions and Formulations

In some embodiments, the IRMA, e.g., toxicity-targeting agents are provided as a composition or formulation, such as a pharmaceutical composition or formulation. Such compositions can be used in accord with the provided methods, such as in an early intervention for the prevention, treatment or amelioration of a toxicity, such as to delay, attenuate, reduce viral infection induced-CRS or organ toxicity in the subject.

In some embodiments, the IRMA are formulated with a pharmaceutical carrier.

In some embodiments, the pharmaceutical composition can contain preservatives.

Buffering agents in some aspects are included in the compositions.

In some embodiments, the agents are administered in the form of a salt, e.g., a pharmaceutically acceptable salt.

Active ingredients may be entrapped in microcapsules, in colloidal drug delivery systems (for example, liposomes, albumin microspheres, microemulsions, nano-particles and nanocapsules) or in macroemulsions.

The pharmaceutical composition in some aspects can employ time-released, delayed release, and sustained release delivery systems such that the delivery of the composition occurs prior to, and with sufficient time to cause, sensitization of the site to be treated.

The agents can be administered by any suitable means, for example, by bolus infusion or by injection.

The agents may be administered using standard administration techniques, formulations, and/or devices. Provided are formulations and devices, such as syringes and vials, for storage and administration of the compositions. When administering a therapeutic composition (e.g., a pharmaceutical composition containing an agent that treats or ameliorates symptoms of a toxicity, such as viral infection induced-CRS or organ toxicity), it will generally be formulated in a unit dosage injectable form (solution, suspension, emulsion).

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition.

### EXAMPLES

### Use of Lck Inhibitors (Dasatinib, Ponatinib, Bosutinib) in the Prevention and Treatment of Viral Infection Associated Complications

An 80 year old male gets infected with SARS-cov2 in a nursing home. Patient develops persistent fever lasting 12 hours, tachycardia, tachypnea, increased oxygen requirement and pulmonary infiltrate accompanied by elevation of CRP (20 mg/L) and IL6 (15 pg/ml). Patient is administered intravenous fluids and supplement oxygen without significant improvement. Patient is then administered Dasatinib 70 mg PO daily on day 1 which is gradually increased to 140 mg PO daily over the next 3 days with stabilization of oxygen requirement. The patient tolerates the medication well and does not require ventilatory support after initiation of Dasatinib. In an alternate embodiment, the patient is given Ponatinib (30 mg PO every day) or Bosutinib (600 mg PO every day).

### Use of Lck Inhibitors (Dasatinib, Ponatinib, Bosutinib) in the Prevention of Viral Infection Associated Complications

There is an outbreak of SARS-cov2 in a nursing home. A pilot study is conducted to test the safety and efficacy of administration of Dasatinib 70 mg PO daily in preventing COVID-19 pneumonia and other complications among residents who are asymptomatic or minimally symptomatic but are positive for SARS-cov2 on PCR. In an alternate embodiment, the subjects are given Ponatinib (30 mg PO every day) or Bosutinib (600 mg PO every day).

### Use of IRMA in the Prevention and Treatment of Viral Infection Associated Complications

A 70 year old female with diabeter gets infected with SARS-cov2. Patient develops grade 1 CRS as reflected by fever of 38.5° C. but has normal blood pressure and normal saturation of O2 as measured by pulse oximetry. Patient is not hypoxic. Patient is then administered Dasatinib 70 mg PO daily on day 1 which is gradually increased to 140 mg PO daily on day 3 and then continued daily at 140 mg PO dose. The patient tolerates the medication well and does not require supplementation oxygen after initiation of Dasatinib. In an alternate embodiment, the patient is given Ponatinib (30 mg PO every day) or Bosutinib (600 mg PO every day) or Tocilizumab (8 mg/kg i.v.) or Sarilumab (200 mg subcutaneously every 2 weeks), or Siltuximab (11 mg/kg i.v.) instead of Dasatinib. In an alternate embodiment, the patient is also given Lopinavir/Ritonavir (100/50 mg every four hours) or is given Remdesivir or Favilavir at the recommended doses.

### Use of Lck Inhibitors (Dasatinib, Ponatinib, Bosutinib) in the Prevention and Treatment of Viral Infection Associated Complications

A 70 years old female with diabetes mellitus gets infected with SARS-cov2. Patient develops grade II CRS as reflected by persistent fever of greater than 38.5° C. for 24 hours, low blood pressure and low saturation of O2 (<90% on room air) as measured by pulse oximetry. Patient is then administered Dasatinib 140 mg PO daily within 4 hours of developing grade 2 CRS with stabilization. The patient tolerates Dasatinib well. In an alternate embodiment, the patient is given Ponatinib (30 mg PO every day) or Bosutinib (600 mg PO every day) or Tocilizumab (8 mg/kg i.v. ×1 repeated at 1 week interval if needed) or Sarilumab (200 mg subcutaneously every 2 weeks), or Siltuximab (11 mg/kg i.v time 1 and repeated at 1 week interval if needed.) instead of Dasatinib. In an alternate embodiment, the patient is also given prednisone 60 mg orally every day.

### Use of Lck Inhibitors (Dasatinib, Ponatinib, Bosutinib) in the Prevention and Treatment of Viral Infection Associated Complications

A 70 year old male gets infected with SARS-cov2 in a nursing home. Patient develops persistent fever lasting 24 hours, along with elevation of CRP (20 mg/L) and IL6 (15 pg/ml). Patient is administered intravenous fluids, vasopressin and supplement oxygen with a face mask without significant improvement. Patient is then administered Dasatinib 140 mg PO daily and Remdesivir with stabilization of oxygen requirement. The patient tolerates the medications well and does not require ventilatory support after initiation of Dasatinib. In an alternate embodiment, the patient is given Ponatinib (30 mg PO every day) or Bosutinib (600 mg PO every day) instead of Dasatinib and Remdesivir is replaced by Favilavir. In another alternate embodiment, the patient is given Dasatinib plus hydroxychloroquine 200 mg thrice daily and azithromycin 250 mg once a day.

### Use of Lck Inhibitors (Dasatinib, Ponatinib, Bosutinib) in the Prevention and Treatment of Viral Infection Associated Complications

An 80 year old male gets infected with Influenza A in a nursing home. Patient develops persistent fever, tachycardia, tachypnea, increased oxygen requirement and pulmonary infiltrate accompanied by elevation of CRP (20 mg/L) and IL6 (15 pg/ml). Patient is administered intravenous fluids and supplement oxygen without significant improvement. Patient is then administered Dasatinib 70 mg PO daily on day 1 which is gradually increased to 140 mg PO daily over the next 3 days with stabilization of oxygen requirement. The patient tolerates the medication well and does not require ventilatory support after initiation of Dasatinib. In an alternate embodiment, the patient is given Ponatinib (30 mg PO every day) or Bosutinib (600 mg PO every day). Finally, in an alternate embodiment, the patient is also administered prednisone 60 mg PO every day along with LPV/r 400/100 mg twice daily and Eculizumab (900 mg IV).

### Use of C5-Inhibitor Eculizumab in the Prevention and Treatment of Viral Infection Associated Complications

A 70 year old male gets infected with influenza virus A in a nursing home. Patient develops ARDS and shock manifested by worsening renal function, oliguria, hypotension, fever, tachycardia, tachypnea, increased oxygen requirement and altered mental status. Patient is administered intravenous fluids and supplement oxygen without significant improvement. Patient is then administered Tocilizumab (600 mg i.v.) on day 5 after transplantation with stabilization of blood pressure. However, patient continues to have renal and neurological deterioration with worsening mental status and confusion. Subsequently, the patient is administered Ecoluzimab at the recommended dose (900 mg intravenously) on day 6 after stem cell transplant with gradual improvement in mental function. The dose of Ecoluzimab is repeated at weekly interval depending on the discretion of the physician.

### Use of C5-Inhibitor Ravulizumab (ALXN1210) in the Prevention and Treatment of Viral Infection Associated Complications

A 70 years old male patient develops infection with SARS-cov2. The patient develops persistent fever of >38.5° C. for 6 hours and O2 saturation of 92% on room air. His CRP levels doubles in 24 hours. The patient is administered Tocilizumab at 8 mg/Kg by intravenous infusion. The patient's condition stabilizes initially but he shows deterioration of mental function, including confusion, which is accompanied by gradual elevation of LDH over the next week which is accompanied by fall in Haptoglobin and appearance of schistiocytes on peripheral smear. Patient is administered Ravulizumab at the recommended dose (3000 mg i.v.) with gradual improvement in mental function.

### Emergency Administration of an IRMA (e.g. Tocilizumab) Using Autoinjector

A 50 years old male patient with history of chronic obstructive pulmonary disease and diabetes mellitus is diagnosed with infection with SARS-cov2 in the middle of a pandemic. Due to overcrowding in the hospital, the patient is monitored at home in a rural area and is provided an auto-injector containing Tocilizumab (8 mg/Kg). On day 10 after the infection, the patient's condition worsens and he develops progressive worsening shortness of breath with oxygen saturation falling to 87% on room air as measured by Pulse-oximeter. The patient is instructed over the phone to administer Tocilizumab using the auto-injector with stabilization. Patient tolerates the medication well.

### Example: Self Administration of an IL6/IL6R Antagonist by Subcutaneous Route in at Home Setting

A 50 years old female patient with history of chronic obstructive pulmonary disease and hypertension is diagnosed with infection with SARS-cov2 during the middle of a pandemic. Due to overcrowding in the hospital, the patient is monitored in her home in a remote rural area. On day 8 after the infection, the patient's condition worsens and she develops progressive worsening of shortness of breath with oxygen saturation falling to 89% on room air as measured by Pulse-oximeter. The patient administers Sarilumab 200 mg by subcutenous injection. Patient tolerates the medication well.

### Use of CRP and IL6 to Identify Patients at Risk of Viral Complications and Candidate for Therapy with IRMA

A retrospective review is conducted to identify patients at high risk of developing complications form virus infections (e.g., SARS-Cov2, SARS etc.). It is observed that patients with elevated level of serum CRP (CRP>20 mg/L or doubling of CRP within 24 h), or elevated level of serum IL6 (IL6>5 pg/ml or doubling of IL6 within 24 h) are at high risk of complications from a virus infection and have better outcome when treated with an IRMA.

### A Clinical Trial to Study the Effect of Lck Inhibitors in Combination with Antivirals in Patients with COVID-19

### Study Design

Interventional, open-label, phase I/II prospective trial of participants with mild to moderate COVID-19 infection requiring hospital admission.

In Part 1 (preliminary safety/feasibility phase), 30 participants will receive dasatinib (n=10), ponatinib (n=10), or Bosutinib (n=10) alone in combination with other agents selected from Remdesivir (RDV), lopinavir/ritonavir (LPV/r, Kaletra), hydroxychloroquine (HC), hydroxychloroquine plus azithromycin, chloroquine and Favilavir.

In Part 2 (randomized phase), 100 participants will be randomized 1:1 to dasatinib vs placebo (or no therapy if placebo is not available) plus selected antiviral therapy selected from Remdesivir, lopinavir/ritonavir (LPV/r, Kaletra), hydroxychloroquine (HC), hydroxychloroquine plus azithromycin, chloroquine, Oseltamivir Phosphate, ribavarine, and XOFLUZA™ (baloxavir marboxil), Favilavir.

### Objectives

Primary. To assess the efficacy of Lck inhibitors (e.g., dasatinib, ponatinib or Bosutinib) combined with antiviral therapy in patients with COVID-19-related mild and moderate disease in terms of reduction of the percentage of subjects requiring ICU admission.

Inclusion criteria.
Age 18 years or greater
Positive PCR for SARS-CoV2 in a respiratory tract sample
Persistent Fever of >38.5° C. for more than 4 hours
Cough
Peripheral capillary oxygen saturation (SpO2)>93% on room air
Willing and able to provide written informed consent prior to performing study procedures

### Treatment Plan

Part 1: Open-label dasatinib 140 mg/day/orally, Ponatinib 30 mg/day/orally, or Bosutinib 200 mg/day/orally alone or plus selected other therapy for up to 30 days.

Part 2: 140 mg/day/orally or Ponatinib 30 mg/day/orally, or Bosutinib 200 mg/day/orally vs dasatinib placebo (or no therapy if placebo is not available) plus selected antiviral therapy for up to 30 days

Selected other therapy: The investigator, in collaboration with the participant's treating physician, will choose from one of the following: 1) HC 200 mg thrice daily (co-administration with azithromycin 250 mg is permitted), 2) Remdesivir (RDV) at either the dosage required by the Gilead expanded-access program, or the FDA-approved dosage (when licensed), 3) LPV/r 400/100 mg twice daily, 4) Oseltamivir Phosphate 75 mg PO qday 4) another antiviral agent for which promising preliminary data becomes available during the course of the trial.

### Definitions

Recovery. Survival to hospital discharge

### Outcome Measures

Primary. The percentage of patients requiring transfer to ICU.

Secondary.
The percentage of patients achieving surviving until hospital discharge
Total WBC count, absolute lymphocyte count and percentage, haemoglobin, creatinine, ALT and AST, SARS-CoV2 qualitative PCR, ACE2 levels, CRP, d-dimer, LDH, cytokine signatures including IL-6 and TNFα values at baseline and days 3, 7, 14; mortality.

### Clinical Assessment

All enrolled subjects will be assessed daily for clinical parameters including temperature, respiratory rate, pulse rate, blood oxygen saturation, and blood pressure. Chest X-ray, pulmonary computed tomography (CT) will be performed at baseline, weeks 1 and 2.

### A Clinical Trial to Study the Effect of Lck Inhibitors in Patients with COVID-19

### Study Design

Interventional, open-label, phase I prospective trial of participants with mild to moderate COVID-19 infection requiring hospital admission.

In Part 1 (preliminary safety/feasibility phase), 30 participants will receive dasatinib (n=10), ponatinib (n=10), or Bosutinib (n=10) alone in combination with other agents selected from Remdesivir (RDV), lopinavir/ritonavir (LPV/r, Kaletra), hydroxychloroquine (HC), hydroxychloroquine plus azithromycin, chloroquine,

### Objectives

Primary. To assess the efficacy of Lck inhibitors (e.g., dasatinib, ponatinib or Bosutinib) combined with antiviral therapy in patients with COVID-19-related mild disease in terms of reduction of the percentage of subjects requiring ICU admission.

Inclusion criteria.
Age 18 years or greater; Positive PCR for SARS-CoV2 in a respiratory tract sample; Cough and/or pneumonia on chest imaging; SpO2>92% on room air

### Treatment Plan

Part 1: Open-label dasatinib 140 mg/day/orally, Ponatinib 30 mg/day/orally, or Bosutinib 200 mg/day/orally alone or plus selected other therapy for up to 30 days.

Part 2: 140 mg/day/orally or Ponatinib 30 mg/day/orally, or Bosutinib 200 mg/day/orally vs dasatinib placebo (or no therapy if placebo is not available) plus selected antiviral therapy for up to 30 days

Selected other therapy: The investigator, in collaboration with the participant's treating physician, will choose from one of the following: 1) HC 200 mg thrice daily (co-administration with azithromycin 250 mg is permitted), 2) Remdesivir (RDV) at either the dosage required by the Gilead expanded-access program, or the FDA-approved dosage (when licensed), 3) LPV/r 400/100 mg twice daily, 4) Oseltamivir Phosphate 75 mg PO qday 4) another antiviral agent for which promising preliminary data becomes available during the course of the trial.

### Definitions

Recovery. Survival to hospital discharge

### Outcome Measures

Primary. The percentage of patients requiring transfer to ICU.

### Compositions and Method for Prevention of Viral Infections in a Subject

Modulation of tissue microenvironment for prevention of viral infections. The mucosal epithelial cells lining the upper respiratory track (e.g., nasal and pharyngeal mucosa) serves as the portal of entry for upper respiratory tract viruses, such as common cold viruses, rhino-virus, adenoviruses, influenza A, influenza B, parainfluenza virus, human respiratory syncytial virus, human coronavirus (e.g., SARS, COVID-19 and MERS), enteroviruses and human metapneumovirus. Infection of the lining epithelium results in production of inflammatory cytokines that provide a favorable microenvironment for viral replication and spread. Although measures such as hand-hygiene and masks are used to prevent the spread of upper respiratory viruses, they are frequently inadequate. The present disclosure describes the use of compositions and methods to prevent upper respiratory tract viral infections by modulating the local microenvironment of the upper respiratory tract so as to make it less favorable for the replication of upper respiratory viruses.

The present disclosure also describes the use of compositions and methods to decrease the infectivity of a subject infected with an upper respiratory tract viral infections by modulating the local microenvironment of the upper respiratory tract so as to make the subject less infectious to others.

In one embodiment, the method involves changing the local pH of the upper respiratory tract. In another embodiment, the method involves changing the ionic composition of the upper respiratory tract. In an exemplary embodiment, the pH of the upper respiratory tract can be changed by frequent rinses with alkaline solutions (i.e., pH>7.4). Exemplary alkaline solutions include ethyl alcohol. In an exemplary embodiment, the subject at risk of an upper respiratory tract infection does oral gargles with a solution containing 10-70% ethanol every 5 minutes, 10 minutes, 15 minutes, 30 minutes, 1 hour, 2 hour, 4 hour, 6 hour, 8 hour, 12 hour, 16 hour and 24 hour. In an exemplary embodiment, the above regimen is continued for as long as the subject is at risk of contacting the infection or at risk of spreading the infection. In an exemplary embodiment, the above regimen is continued for more than 1 days, 2 days, 5 days, 7 days, 10 days, 14 days, 21 days, 30 days and the like.

In an exemplary embodiment, the subject at risk of an upper respiratory tract infection does oral gargles with a Listerine every 5 minutes, every 10 minutes, every 15 minutes, every 30 minutes, every 1 hour, every 2 hour, every 4 hour, every 6 hour, every 8 hour, every 12 hour, every 16 hour and every 24 hours. In an exemplary embodiment, the above regimen is continued for as long as the subject is at risk of contacting the infection. In an exemplary embodiment, the above regimen is continued for more than 1 days, 2 days, 5 days, 7 days, 10 days, 14 days, 21 days, 30 days and the like.

In an exemplary embodiment, the method of preventing an upper respiratory tract infection involves having the subject at risk of an upper respiratory tract infection do oral gargles with a solution containing Ozonated Aqua, Finnish Birch Xylitol, Sodium Chlorite, Sodium Bicarbonate, Zinc Citrate, Menthol every 5 minutes, 10 minutes, 15 minutes, 30 minutes, 1 hour, 2 hour, 4 hour, 6 hour, 8 hour, 12 hour, 16 hour and 24 hour. In an exemplary embodiment, the method of preventing an upper respiratory tract infection involves having the subject at risk of an upper respiratory tract infection do oral gargles with a solution containing one or more of Sodium Chlorite, Sodium Bicarbonate, Zinc Citrate and Menthol every 5 minutes, 10 minutes, 15 minutes, 30 minutes, 1 hour, 2 hour, 4 hour, 6 hour, 8 hour, 12 hour, 16 hour and 24 hour. In an exemplary embodiment, the method of preventing an upper respiratory tract infection involves having the subject at risk of an upper respiratory tract infection do oral gargles with a solution containing Hydrogen peroxide every 5 minutes, 10 minutes, 15 minutes, 30 minutes, 1 hour, 2 hour, 4 hour, 6 hour, 8 hour, 12 hour, 16 hour and 24 hour. In an exemplary embodiment, the above regimen is continued for as long as the subject is at risk of contacting the infection. In an exemplary embodiment, the above regimen is continued for more than 1 days, 2 days, 5 days, 7 days, 10 days, 14 days, 21 days, 30 days and the like.

In an exemplary embodiment, the subject takes zinc lozenges (one lozenge every 1 hours while awake) containing 13.3 mg of zinc from zinc gluconate for more than 1 days, 2 days, 5 days, 7 days, 10 days, 14 days, 21 days, 30 days and the like.

The above measures can be also used to prevent the spread of viral infection, including SARS-CoV2 to others from a subject already infected with the virus.

1. A method of treatment, comprising administering to a subject an Immune response modulating agent (IRMA) and/or other treatment capable of treating, preventing, delaying, or attenuating the development of complications of a viral infection, wherein, at the time of said administration, the subject has a viral infection, a suspected viral infection or an exposure to a viral pathogen, and wherein:
(a) the administration of the IRMA or other treatment is:
(i) at a time that is less than ten day(s) after development of viral infection or exposure to a viral infection; and/or
(ii) at a time at which the subject does not exhibit a sign or symptom of severe cytokine release syndrome (CRS) and/or does not exhibit grade 2 or higher viral infection induced-CRS; and/or
(iii) at a time at which the subject does not exhibit a sign or symptom of severe organ toxicity and/or does not exhibit grade 2 or higher organ toxicity; and/or
(b) between the time of development of viral infection or exposure to a viral infection and the time of the administration of the IRMA or other treatment (i) the subject has not exhibited severe viral infection induced-CRS and/or has not exhibited grade 2 or higher viral infection induced-CRS and/or (ii) the subject has not exhibited severe organ toxicity and/or does not exhibit grade 2 or higher organ toxicity; and/or
(c) the viral infection is selected from any one or more of coronavirus, influenza A, influenza B, parainfluenza, Respiratory syncytial virus (RSV), or an enterovirus.

2. (canceled)

3. The method of claim 1, wherein the IRMA or other treatment is administered at a time at which the subject exhibits a sign or symptom of viral infection induced-CRS and/or exhibits grade 1 viral infection induced-CRS or is administered within 24 hours after the subject exhibits a first sign or symptom of grade 1 viral infection induced-CRS following the development of first sign, symptom or laboratory evidence of a viral infection.

4. The method of claim 1, wherein:
a) the sign or symptom of grade 1 viral infection induced-CRS is a fever; and/or the IRMA or other treatment is administered within 24 hours after the first sign of a fever; and/or
b) the sign or symptom of grade 1 viral infection induced-CRS is a cough; and/or the IRMA or other treatment is administered within 24 hours after the first sign of a cough; and/or
c) the sign or symptom of grade 1 viral infection induced-CRS is a shortness of breath; and/or the IRMA or other treatment is administered within 24 hours after the first sign of a shortness of breath; and/or
d) the sign or symptom of grade 1 viral infection induced-CRS is a chest pain; and/or the IRMA or other treatment is administered within 24 hours after the first sign of a chest pain; and/or
e) the sign or symptom of grade 1 viral infection induced-CRS is headache; and/or the IRMA or other treatment is administered within 24 hours after the first sign of headache; and/or
f) the sign or symptom of grade 1 viral infection induced-CRS is tachypnea (respiratory rate greater than 12 per minute); and/or the IRMA or other treatment is administered within 24 hours after the first sign of tachypnea; and/or
g) the sign or symptom of grade 1 viral infection induced-CRS is hypoxia (O2 saturation of less than 92% on room air); and/or the IRMA or other treatment is administered within 24 hours after the first sign of hypoxia; and/or
h) the sign or symptom of grade 1 viral infection induced-CRS is elevation of CRP (CRP greater than 1.5× upper limit of normal); and/or the IRMA or other treatment is administered within 24 hours after the first elevation of CRP of greater than 1.5× the upper limit of normal; and/or
i) the sign or symptom of grade 1 viral infection induced-CRS is elevation of IL6 (IL6 greater than 1.5× upper limit of normal); and/or the IRMA or other treatment is administered within 24 hours after the first elevation of IL6 of greater than 1.5× the upper limit of normal; and/or
j) the sign or symptom of grade 1 viral infection induced-CRS is doubling of CRP (doubling of CRP in a 24 h period); and/or the IRMA or other treatment is administered within 24 hours after the first doubling of CRP in a 24 h period; and/or
k) the sign or symptom of grade 1 viral infection induced-CRS is doubling of serum IL6 in a 24 hour period; and/or the IRMA or other treatment is administered within 24 hours after the first doubling of serum IL6 in a 24 hour period; and/or
l) the sign or symptom of grade 1 viral infection induced-CRS is elevation of CRP greater than 20 mg/L; and/or the IRMA or other treatment is administered within 24 hours after the first elevation of CRP of greater than 20 mg/L; and/or
m) the sign or symptom of grade 1 viral infection induced-CRS is elevation of IL6>50 pg/ml; and/or the IRMA or other treatment is administered within 24 hours after the first elevation of IL6 of greater than 50 pg/ml, and/or
n) the sign or symptom of grade 1 viral infection induced-CRS is radiological or clinical evidence pulmonary infiltrate; and/or the IRMA or other treatment is administered within 24 hours after the first radiological or clinical evidence of pulmonary infiltrate; and/or
o) the sign or symptom of grade 1 viral infection induced-CRS is lymphopenia of <20% of WBC; and/or the IRMA or other treatment is administered within 24 hours after the first laboratory evidence of lymphopenia.

5-6. (canceled)

7. The method of claim 1, wherein the IRMA or other treatment is administered within about 16 hours, within about 12 hours, within about 8 hours, within about 2 hours or within about 1 hour after the first sign of a fever, cough, malaise, shortness of breath, chest pain, headache, hypoxia, elevation of CRP, elevation of IL6, pulmonary infiltrates on clinical or radiological examination and/or need for supplemental oxygenation.

8. The method of claim 7, wherein the fever is a sustained fever, where cough is a sustained cough, where malaise is a sustained malaise, where shortness of breath is a sustained shortness of breath, where hypoxia, elevation of CRP, elevation of IL6, lymphopenia, pulmonary infiltrates on clinical or radiological examination and/or need for supplemental oxygenation are sustained for more than 4 hours.

9. The method of claim 8, wherein the fever is a fever that is not reduced or not reduced by more than 1° C. after treatment with an antipyretic and/or wherein the fever has not been reduced by more than 1° C., following treatment of the subject with an antipyretic.

10. The method of claim 9, wherein the fever comprises a temperature of at least or at least about 38.0° C.

11. The method of claim 10, wherein:
the fever comprises a temperature that is between or between about 38.0° C. and 42.0° C., 38.0° C. and 39.0° C., 39.0° C. and 40.0° C. or 40.0° C. and 42.0° C., each inclusive; or
the fever comprises a temperature that is greater than or greater than about or is or is about 38.5° C., 39.0° C., 39.5° C., 40.0° C., 41.0° C., 42.0° C.; or
the elevation of CRP comprises CRP greater than about 10 mg/L; or
the elevation of IL6 comprises IL6 greater than about 30 pg/ml; or
Lymphopenia comprises lymphocyte % of less than about 20% of WBC; or
oxygen saturation in arterial blood is less than about 93%.

12. The method of claim 1, wherein the IRMA or other treatment is administered less than ten days after development of first sign, symptom or lab evidence of viral infection, less than eight days after development of first sign, symptom or lab evidence of viral infection, less than six days after development of first sign, symptom or lab evidence of viral infection, less than 4 days after development of first sign, symptom or lab evidence of viral infection, less than 2 days after development of first sign, symptom or lab evidence of viral infection.

13. The method of claim 1, wherein the viral infection comprises infection with a coronavirus, influenza A, influenza B, parainfluenza, Rhinovirus, RSV, or enterovirus or a combination.

14. The method of claim 1, wherein the IRMA or other treatment is or comprises a steroid, an Src/Lck inhibitor, C5 inhibitor, or an antagonist or inhibitor of a cytokine receptor or cytokine selected from the group consisting of IL-10, IL-10R, IL-6, IL-6 receptor, IFNγ, IFNGR, IL-2, IL-2R/CD25, MCP-1, CCR2, CCR4, MIPIβ, CCR5, TNFalpha, TNFR1, IL-1, and IL-lRalpha/IL-lbeta.

15. The method of claims 14, wherein the antagonist or inhibitor is or comprises an agent selected from among an antibody or antigen-binding fragment, a small molecule, a protein or peptide and a nucleic acid.

16. The method of claim 15, wherein the IRMA or other treatment is or comprises an agent selected from the group consisting of Dasatinib, Ponatinib, Bosutinib, tocilizumab, situximab, sarilumab, olokizumab (CDP6038), elsilimomab, ALD518/BMS-945429, sirukumab (CNTO 136), CPSI-2634, ARGX-109, FE301, FM101, tesidolumab, and eculizumab.

17. The method of claim 1, wherein the IRMA or other treatment is or comprises Dasatinib.

18. The method of claims 17, where dasatinib is administered orally at a dose of at least 10 mg/day.

19. The method of claim 1, wherein the IRMA or other treatment is or comprises Ponatinib.

20. The method of claim 19, where Ponatinib is administered orally at a dose of at least 5 mg/day.

21. The method of claim 1, wherein the IRMA or other treatment is or comprises Bosutinib.

22. The method of claim 21, where Bosutinib is administered orally at a dose of at least 50 mg/day.

23. The method of claim 1, wherein the IRMA or other treatment is or comprises Tocilizumab.

24. The method of claim 23, wherein the tocilizumab is administered in a dosage amount of from about 1 mg/kg to 10 mg/kg.

25. The method of claim 1, wherein the IRMA or other treatment is or comprises Sarilumab (Kevzara).

26. The method of claim 25, wherein Sarilumab is administered in a dosage amount of about 20 mg, each inclusive or Sarilumab is administered in a dosage amount of at least about 50 mg subcutaneously once every 1-2 weeks.

27. The method of claim 1, wherein the IRMA or other treatment is or comprises Siltuximab.

28. The method of claim 27, wherein Siltuximab is administered in a dosage amount of about 1 mg/kg to 11 mg/kg, each inclusive, or the tocilizumab is administered in a dosage amount of at least about 2 mg/kg.

29. The method of claim 1, wherein the IRMA or other treatment is or comprises eculizumab.

30. The method of claim 29, wherein eculizumab is administered in a dosage amount of about 500-900 mg intravenously and repeated every week if needed.

31. The method of claim 1, further comprising administering a second agent, which optionally is a steroid, to the subject, wherein the steroid is administered:
(i) at a time that is within 9 days after development of first sign, symptoms of lab evidence of a viral infection,
(ii) at a time that is within 24 hours after the first sign of hypoxia after development of first sign, symptoms of lab evidence of a viral infection,
(iii) at a time at which the subject exhibits grade 2 cytokine release syndrome (viral infection induced-CRS) or within 24 hours after the subject exhibits a first sign of grade 2 viral infection induced-CRS following development of first sign, symptoms of lab evidence of a viral infection; and/or
(iv) at a time at which the subject exhibits grade 2 organ toxicity or within 24 hours after the subject exhibits a first sign or symptom of grade 2 organ toxicity following development of first sign, symptoms of lab evidence of a viral infection.
(v) at a time that is within 24 hours after the first sign, symptom or radiological evidence of pulmonary infiltrate after development of first sign, symptoms of lab evidence of a viral infection.

32. (canceled)

33. The antiviral agent of claim 31, where the second agent is an antiviral agent selected from the group consisting of lopinavir/ritonavir (LPV/r, Kaletra), remdesivir, Favilavir, Oseltamivir Phosphate, ribavarine, and XOFLUZA™ (baloxavir marboxil).

34. The method of claim 1, further comprising administering a second agent, which is selected from the group of Chloroquine, hydroxychloroquine (HC) and JAK inhibitor.
